(19) United States
(12) Patent Application Publication
(10) Pub. No.: US 2009/0264884 A1
(43) Pub. Date: Oct. 22, 2009

(54) Apparatus for External Fixation of the Pelvic Ring

(76) Inventors: Alessandro Masse, Torino (IT); Domenico Aloj, (Torino) (IT); Antonio Biasibetti, Torino (IT)

Correspondence Address: COLEMAN SUDOL SAPONE, P.C., 714 COLORADO AVENUE, BRIDGE PORT, CT, 06605-1601, US

(21) Appl. No.: 11/989,357

(22) PCT Filed: Jul. 25, 2006
(86) PCT No.: PCT/EP2006/007321

§ 371 Date: Jan. 24, 2008

(30) Foreign Application Priority Data
Jul. 28, 2005 (IT) PD2005 A000232

(51) Int. Cl. A61B 17/60

(52) U.S. Cl. 606/59; 606/54

(57) ABSTRACT

An apparatus for external fixation of the pelvic ring, having left grip means and right grip means to be engaged in insertion/engagement holes formed respectively in the left and right iliac bone of the pelvic ring, left locking means and right locking means for mutually locking respectively the left grip means and the right grip means, left articulation means and right articulation means in order to allow to connect articulately the left and right locking means, and transabdominal means for joining the left and right articulation means. The apparatus has left and right means for guiding drilling means used by the orthopedic surgeon to provide the insertion/engagement holes so as to allow correct execution of the insertion/engagement holes.

The present invention relates to an apparatus for external fixation of the pelvic ring, for the surgical and orthopedic treatment of fractures of the human pelvis.

Conventional apparatuses for external fixation of the pelvic ring, used in the current practice of surgical orthopedic procedures suitable to achieve reduction of pelvic fractures, generally comprise left and right grip means that the surgeon inserts and engages by screwing in insertion/engagement holes provided in the left and right iliac bone. Those apparatuses also include left and right locking means, that respectively lock the left and right grip means together. Those conventional apparatuses are provided with left and right articulation means for articulating the left and right locking means. Those conventional apparatuses include joining means, which are required in order to provide the transabdominal joining of the left and right articulation means.

The procedure for applying those apparatuses, during orthopedic surgical procedures for reducing pelvic fractures, generally comprises the following steps.

In a first preliminary step, the orthopedic surgeon determines the positions on the left and right iliac bone where the insertion/engagement holes for the grip means are to be provided by means of a palpatory examination of the region of application of the apparatus.

In a second preliminary step, the surgeon incises the skin of the patient, in the region of application of the apparatus, at the positions determined earlier, enough to expose the edges of the left and right iliac bone. In a third preliminary step, the orthopedic surgeon performs, by using traditional drilling means, the holes for insertion/engagement on the left and right iliac bone. In a first application step, the surgeon inserts the grip means in the insertion/engagement holes and engages them by screw coupling therein. In a second application step, the surgeon locks the left and right grip means to the left and right locking means, respectively. Then, in a third application step, the surgeon provides the connection of the left and right locking means by virtue of the articulation and joining means. At the end of this application procedure, the apparatus forms a solid structure, which temporarily replaces the pelvis until the fractures that affect it have been resolved completely.

The main drawback observed in the prior art apparatuses is that they are unlikely to ensure conditions of fixation of the fractured pelvis that are suitable to provide the abdomen containment action performed by the intact pelvis. This condition causes the onset of the risk of hemorrhages inside the abdomen of the patient, which can seriously endanger his life. This drawback arises from the operating difficulties encountered by the orthopedic surgeon in correctly performing the steps that precede application, which are the most delicate moments of the entire procedure for applying the apparatus. In particular, the preliminary steps for identifying the positions on the left and right iliac bone where the insertion/engagement holes are to be provided and for providing the insertion/engagement holes are particularly critical and depend heavily on the skill of the orthopedic surgeon. The complexity of execution of those steps can be worsened by particular situations in which the surgeon must operate. For example, performing those preliminary steps is particularly difficult in highly overweight patients and when the surgeon is forced to perform the apparatus application procedure in urgent or critically urgent conditions. This complexity of execution entails that the positions and orientation of the grip means tend to be inaccurate and inadequate to achieve, by applying the apparatus, an ideal condition of fixation of the fractured pelvis. This fact is the origin of the drawbacks described above.

The aim of the present invention is to provide an apparatus for external fixation of the pelvic ring that is capable of solving the drawbacks observed in prior art devices.

An object of the invention is to provide an apparatus that can ensure conditions of fixation of the fractured pelvis that are suitable to apply the same abdomen containment action performed by the intact pelvis.

A further object of the invention is to provide an apparatus that allows the procedure for applying the apparatus to become substantially independent of the execution skills of the orthopedic surgeon and of the operating conditions in which the orthopedic surgeon is forced to work.

A further object of the invention is to provide an apparatus that has a simplified structure with respect to prior art apparatuses, so as to contain production costs and the times required for application procedures.

A further object of the invention is to provide an apparatus that can be manufactured predominantly with radiotransparent materials, so as to facilitate the execution of X-rays and analysis of X-ray images.

This aim and these and other objects, which will become better apparent hereinafter, are achieved by an apparatus for external fixation of the pelvic ring, comprising left grip means and right grip means to be engaged in insertion/engagement holes formed respectively in the left and right iliac bone of said pelvic ring, left locking means and right locking means for mutually locking respectively said left grip means and said right grip means, left articulation means and right articulation means adapted to articulately connect said left and right locking means, and transabdominal means for joining said left and right articulation means, characterized in that it comprises left and right means for guiding drilling means used by the surgeon to provide said insertion/engagement holes so as to allow correct execution of said insertion/engagement holes.

Further characteristics and advantages will become better apparent from the description of an embodiment of the invention illustrated by way of example in the accompanying drawings, wherein:

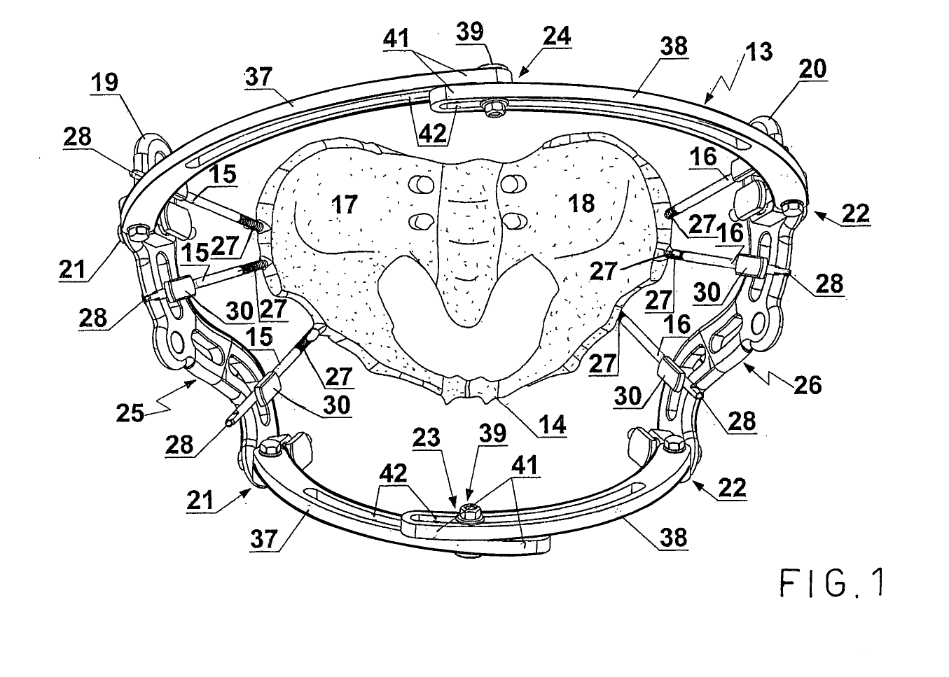

FIG. 1 is a front perspective view of the apparatus according to the invention, in the condition applied to a pelvic ring;

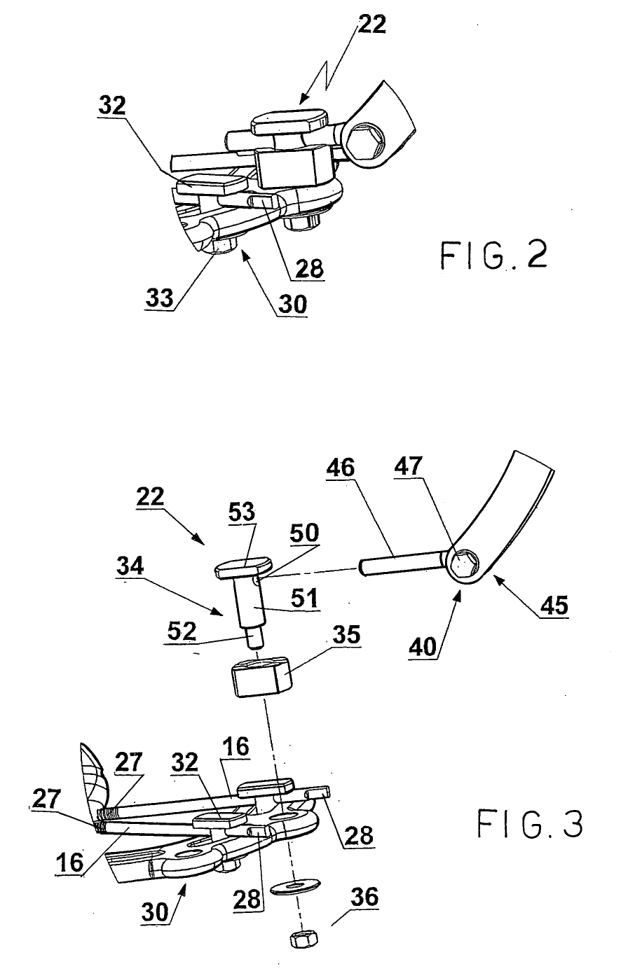

FIG. 2 is a detailed perspective view of the right locking and articulation means of the apparatus;

FIG. 3 is an exploded perspective view of the articulation means shown in FIG. 2;

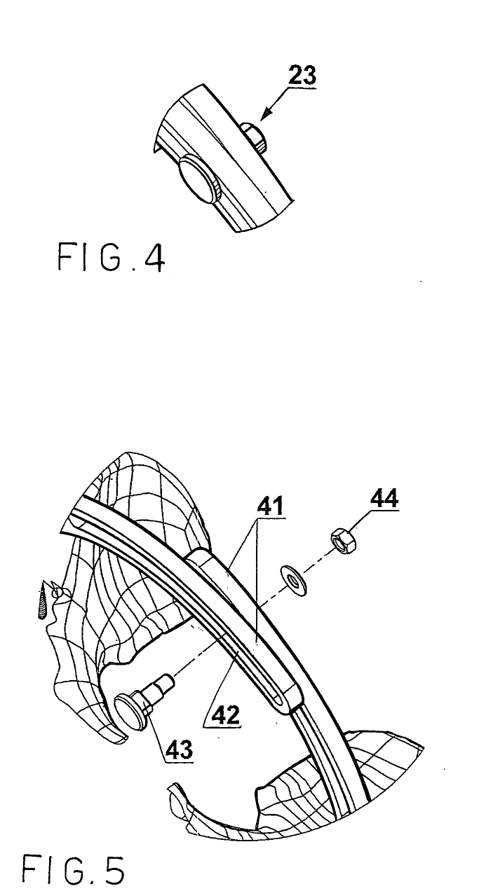

FIG. 4 is a detailed perspective view of the joining means of the apparatus;

FIG. 5 is an exploded perspective view of the joining means of the apparatus;

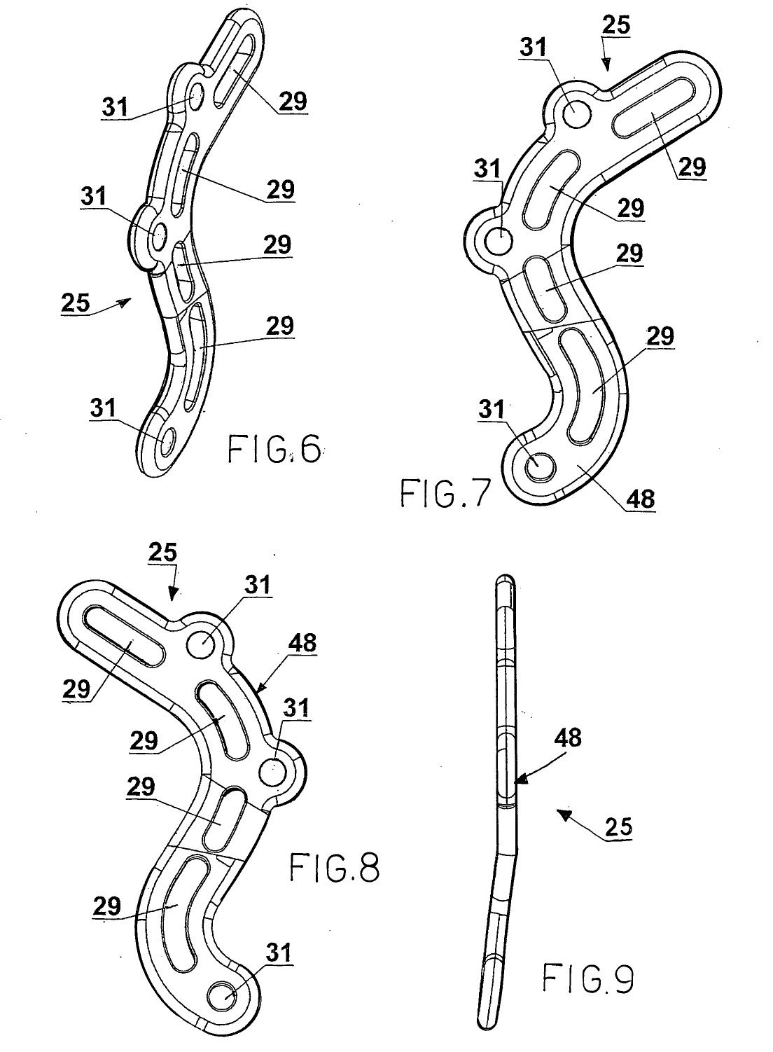

FIGS. 6, 7, 8 and 9 are respectively a perspective view, a front view, a rear view and a side view of the left guiding means of the apparatus;

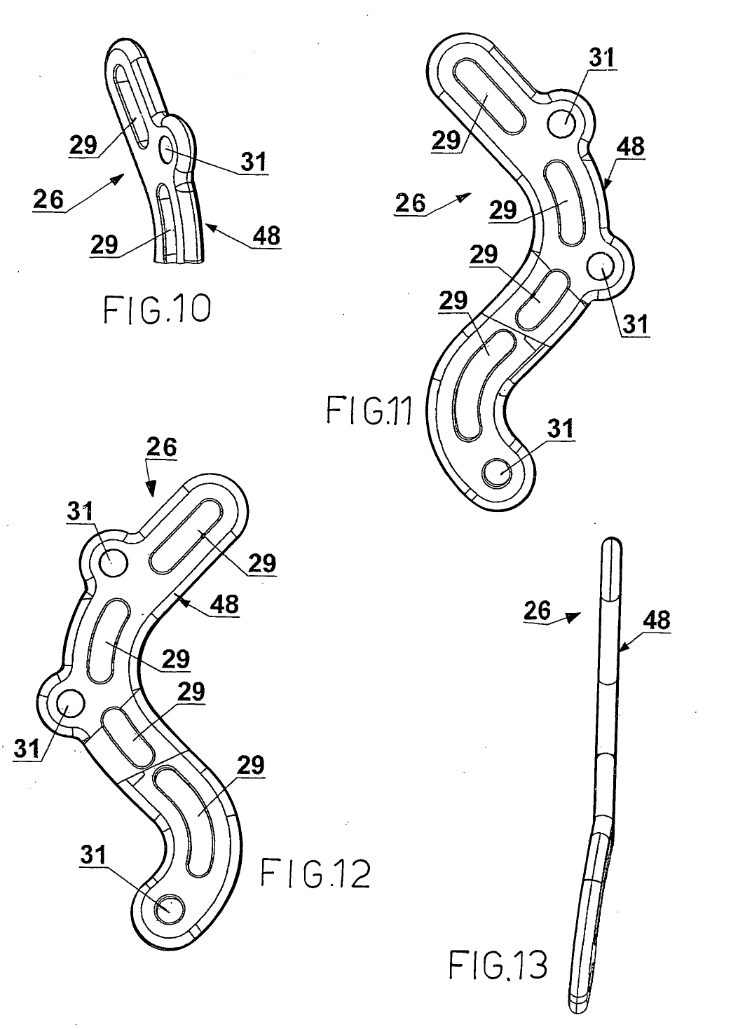

FIGS. 10, 11, 12 and 13 are respectively a perspective view, a front view, a rear review and a side view of the right guiding means of the apparatus.

With reference to the figures, an external fixation apparatus, generally designated by the reference numeral 13, for a pelvic ring 14, comprises left and right grip means 15, 16, which are shown in the condition in which they are engaged in the insertion/engagement holes (not shown) provided respectively in the left and right iliac bone 17, 18 of the pelvic ring 14. The apparatus 13 is provided with left and right locking means, generally designated by the reference numerals 19 and 20, which mutually lock respectively the left and right grip means 15, 16. Left and right articulation means, generally designated by the reference numerals 21 and 22, provide an articulated connection of the left and right locking means 19 and 20. Joining means, generally designated by the reference numerals 23 and 24, join the left and right articulation means 21 and 22 from one side to the other of the abdomen of the patient. Left and right guiding means, generally designated by the reference numerals 25 and 26, are provided for guiding drilling means (not shown), which the orthopedic surgeon uses in producing the insertion/engagement holes. The left and right guiding means 25, 26 allow correct execution of the insertion/engagement holes, independently of the skill of the orthopedic surgeon, as described in greater detail hereinafter.

The left and right grip means 15,16 are formed by rod members, provided at one end with a tip portion provided with an external thread 27, which is inserted and partially engaged by screwing in the insertion/engagement holes and, at the opposite end, with a squared tip portion 28 for coupling to an actuation tool (not shown). The left and right locking means 19, 20 include a series of slots 29, that are provided in the guiding means 25 and 26, fixing members 30, that fix the grip means 15, 16 to the guiding means 25, 26, and pluralities of holes 31, also provided in the guiding means 25 and 26 in which the articulation means 21 and 22 are engaged. A series of slots 29 is formed in the longitudinal central region of the guiding means 25, 26 and a plurality of holes 31 are formed along the peripheral profile of the guiding means 25, 26, which is directed toward the outside of the apparatus 13. The fixing members 30 include screws 32 for engaging the grip means 15, 16 and nuts 33 for locking the engagement screws 32, which are engaged by screw coupling on the engagement screws 32 so as to lock the engagement screws 32 to the guiding means 25, 26.

The engagement screws 32 are formed by a stem which has, at one end, a portion provided with an external thread suitable to mate with the thread provided on the locking nut 33, are provided, on the opposite side with respect to the threaded portion, with a head whose shape is suitable for mating with an actuation tool (not shown), and are provided, proximate to the head, with a transverse hole, which is formed on the stem and is crossed by a grip means 15, 16. The articulation means 21, 22 are provided substantially like the fixing members 30. The articulation means 21, 22 include engagement screws 34 in order to engage the joining means 23 and 24, spacers 35 to be inserted in the engagement screws 34, and locking nuts 36 engaged on the engagement screws 34 in order to fasten them to the guiding means 25, 26. The engagement screws 34 are formed by a stem 51 which has, at one end, a threaded portion 52 which is suitable externally for mating with the thread provided on the locking nut 36, has, on the opposite side with respect to the threaded portion, a head 53 suitable for mating with an actuation tool (not shown), and has a transverse hole 50 formed in the stem proximate to the head, which partially accommodates the joining means 23, 24 namely a tab 46.

The joining means 23, 24 in turn are each constituted by two arc-like members 37 and 38, by adjustable connecting members, generally designated by the reference numerals 39, which mutually connect the joining members 37, 38, allowing to perform an adjustment, and by articulated connecting members, generally designated by the reference numeral 40, which articulately connect the joining members 37, 38 to the articulation means 21, 22. In greater detail, the adjustable connecting members 39 are provided at first ends 41 of the joining members 37, 38 and include two elongated slots 42 provided in the joining members 37 and 38, a locking screw 43 inserted in both slots 42, and finally a fixing nut 44, which is screwed onto the locking screw 43 until the joining members 37, 38 are fixed to each other. The articulated connecting members 40 are provided at second ends 45 of the joining members 37, 38 and include tabs 46, which are connected to the second ends 45 by virtue of articulation screws 47, which allow to articulate the tabs 46 with respect to the joining members 37, 38.

The left and right guiding means 25, 26 include supporting bodies 48, that have a substantially S-shaped curved profile. In detail, this profile substantially reproduces the peripheral shape of the iliac bones 17 and 18 approximately in the central region between the iliac crest and the pubic symphysis, as shown in FIG. 1. To conclude, the locking means 19 and 20, the articulation means 21 and 22, the joining means 23 and 24 advantageously can be made of radiotransparent materials.

The procedure for applying the apparatus 13 to the pelvic ring 14 is described hereafter. As in a conventional procedural technique, the operating procedure can be divided into steps that are preliminary to the application of the apparatus and into steps of application. In a first preliminary step, the surgeon identifies the positions on the crests of the left and right iliac bone 17, 18 where the first holes for insertion/engagement of the first left and right grip means 15, 16 are to be provided, by palpatory examination of the region of application of the apparatus 13. In a second preliminary step, the surgeon incises the skin of the patient, in the region of application of the apparatus 13 at the positions determined earlier, so as to expose the edges of the crests of the left and right iliac bone 17, 18. In a third preliminary step, the surgeon performs, with the aid of traditional drilling means, the holes for insertion/engagement on the crests of the left and right iliac bone 17, 18 at the positions determined earlier. In a first application step, the surgeon inserts the first grip means 15, 16 in the insertion/engagement holes provided earlier and engages them by screwing therein. In a second application step, the surgeon temporarily fits on the first grip means 15, 16 the left and right locking means 19, 20 and the left and right guiding means 25, 26, arranging the profile of the latter in accordance with the external profile of the pelvis 14. In a third application step, the surgeon arranges the drilling means approximately tangent to the internal side of the left and right guiding means 25, 26, with the drilling tool directed toward the region of application of the apparatus 13 in successive positions so as to descend from the crests of the iliac bones 17, 18 at the series of slots 29, so as to determine precisely the additional positions where the other holes for insertion/engagement of the remaining grip means 15, 16 are to be provided. In a fourth application step, the surgeon incises the skin at the positions determined earlier, and by arranging the drilling means as described above produces the remaining insertion/engagement holes, which are mutually oriented correctly. In a fifth step of application, the surgeon inserts and screws the remaining grip means 15, 16 in the insertion/engagement holes that have just been provided and locks the grip means 15, 16 with the left and right locking means 19 and 20. In a sixth application step, the surgeon proceeds with the assembly of the apparatus 13, fixing the left and right articulation means 21, 22 to the locking means 19, 20. In a seventh step, the surgeon completes the assembly of the apparatus 13 by coupling and appropriately fixing the joining means 23, 24 to the articulation means 21, 22.

In practice it has been found that the apparatus achieves the intended aim and objects, overcoming the drawbacks observed in the prior art. The apparatus in fact allows to position and orient the grip means correctly, so as to achieve, at the end of the application of the apparatus, conditions of fixation of the fractured pelvis that are suitable to apply the same abdomen containment action performed by the intact pelvis. With the apparatus according to the present invention, providing the insertion/engagement holes of the grip means is an operation substantially independent of the skills of the surgeon and of the operating conditions. Also, the apparatus has a structure which allows to contain production costs and application times. Finally, the apparatus can be made almost entirely of radiotransparent materials, so as to facilitate both the execution of X-rays exams on the region of application of the apparatus and the analysis of the corresponding results.

This application claims the priority of Italian Patent Application No. PD2005A000232, filed on 28 Jul. 2005, the subject matter of which is incorporated herein by reference.

The apparatus for external fixation of the pelvic ring thus conceived is susceptible of numerous modifications and variations, within the scope of the appended claims. All the details may be replaced with other technically equivalent elements.

In practice, the materials used, so long as they are compatible with the specific use, as well as the contingent shapes and dimensions, may be any according to the requirements and the state of the art.

1. An apparatus for external fixation of the pelvic ring, comprising left grip means and right grip means to be engaged in insertion/engagement holes formed respectively in the left and right iliac bone of said pelvic ring, left locking means and right locking means for mutually locking respectively said left grip means and said right grip means, left articulation means and right articulation means adapted to articulately connect said left and right locking means, and transabdominal means for joining said left and right articulation means, further comprising left and right guiding means for guiding drilling means used by the surgeon to provide said insertion/engagement holes so as to allow correct execution of said insertion/engagement holes.

2. The apparatus according to claim 1, wherein said left and right grip means comprise rod members having, at one end, a tip provided with an external thread intended to be inserted and engaged by screwing at least partially in said insertion/engagement holes and, at the opposite end, a squared tip portion in order to allow mating with an actuation tool.

3. The apparatus according to claim 1, wherein said left and right locking means comprise slots formed in said guiding means, members for fixing said grip means to said guiding means, and holes formed in said guiding means for the engagement of said articulation means.

4. The apparatus according to claim 3, wherein said slots are provided in the longitudinal central region of said guiding means.

5. The apparatus according to claim 3, wherein said holes are formed along the peripheral profile of said guiding means that is directed toward the outside of said apparatus.

6. The apparatus according to claim 3, wherein said fixing members comprise screws for the engagement of said grip means and nuts for locking said screws so as to lock said engagement screws to said guiding means.

7. The apparatus according to claim 6, wherein each of said engagement screws comprises a stem provided, at one end, with a portion having an external thread which is suitable to mate with said locking nut, and, at the opposite end with respect to said threaded portion, with a head whose shape is suitable to mate with an actuation tool and which is provided, proximate to said head, with a transverse hole formed in said stem and crossed by a grip means.

8. The apparatus according to claim 1, wherein said articulation means comprise screws for the engagement of said joining means, spacers to be fitted on said engagement screws, and locking nuts engaged on said engagement screws in order to lock said screws to said guiding means.

9. The apparatus according to claim 8, wherein each of said screws comprises a stem provided, at one end, with a portion having an external thread suitable to mate with said nut, and, at the opposite end, with a head whose shape is suitable to mate with an actuation tool and is provided, proximate to said head, with a transverse hole for partially accommodating the joining means.

10. The apparatus according to claim 1, wherein each of said joining means comprises two arc-like joining members, adjustable connecting members which mutually connect said joining members adjustably, and articulated connection members, which connect articulately said joining members to said articulation means.

11. The apparatus according to claim 10, wherein said adjustable connecting members comprise, at first ends of said connecting members, two elongated slots formed in said connecting members, a locking screw inserted in said slots, and a fixing nut which is screwed onto said screw until said joining members are locked together.

12. The apparatus according to claim 10, wherein said articulated connecting members comprise, at second ends of said joining members, tabs which are connected to said second ends by virtue of articulation screws which allow to articulate said tabs with respect to said joining members.

13. The apparatus according to claim 1, wherein said guiding means comprise supporting bodies which have a substantially S-shaped curved profile.

14. The apparatus according to claim 13, wherein said profile substantially reproduces the peripheral shape of the iliac bones approximately in the central region comprised between the iliac crest and the pubic symphysis.

15. The apparatus according to claim 1, wherein said locking means, said articulation means, said joining means and said guiding means are made of radiotransparent materials.
